(19) United States
(12) Patent Application Publication
(10) Pub. No.: US 2021/0275778 A1
(43) Pub. Date: Sep. 9, 2021

(54) ELONGATE INSTRUMENT AND ELONGATE INSTRUMENT GUARD MEMBER COMPATIBILITY

(71) Applicant: Intuitive Surgical Operations, Inc., Sunnyvale, CA (US)

(72) Inventors: Hans F. Valencia, Santa Clara, CA (US); Peter Fernandez, IV, Menlo Park, CA (US); Lucas S. Gordon, Mountain View, CA (US)

(21) Appl. No.: 17/189,462

(22) Filed: Mar. 2, 2021

Related U.S. Application Data
(60) Provisional application No. 62/984,531, filed on Mar. 3, 2020.

(51) Int. Cl. A61M 25/01; A61M 25/06; A61M 25/00

(52) U.S. Cl. CPC A61M 25/0138; A61M 25/0015; A61M 25/0618

(57) ABSTRACT

A medical tool may comprise sheath and a needle extendable from and retractable into the sheath. The sheath may comprise a tubular shaft member and a guard member attached to the tubular shaft member. The needle may comprise a flexible jacket. One or more of these components may be configured to prevent interference between the flexible jacket and a portion of the guard member as the need is extended or retracted.

### CROSS-REFERENCE TO RELATED APPLICATIONS

This application claims the benefit of and priority to U.S. Provisional Application No. 62/984,531, filed Mar. 3, 2020, which is incorporated by reference herein in its entirety.

### FIELD

Systems for delivering an elongate instrument to a region of interest in a patient anatomy and methods for manufacturing such systems.

### BACKGROUND

Instruments can be used to manipulate and perform tasks in a work space. Such instruments may be configured to be supported and operated partially or entirely by manipulator assemblies. Such instruments and manipulator assemblies can be used to perform non-medical procedures or medical procedures. For example, medical tools or medical manipulators can be used to perform minimally invasive medical procedures. As another example, industrial tools or industrial manipulators can be used in manufacture or testing. As yet other examples, tools or manipulators can be used in procedures for entertainment, exploration, and various other purposes.

Minimally invasive medical techniques may generally be intended to reduce the amount of tissue that is damaged during invasive medical procedures, thereby reducing patient recovery time, discomfort, and harmful side effects. Such minimally invasive techniques may be performed through natural orifices in a patient anatomy or through one or more incisions. Through these natural orifices or incisions clinicians may insert minimally invasive medical instruments (including surgical, diagnostic, therapeutic, imaging, or biopsy instruments) to reach a target tissue location. Some medical and non-medical instruments (including manipulation instruments, imaging instruments, or other sensing instruments, etc.) may be manually operated or may be teleoperated, robotic-assisted, or otherwise computer-assisted. One minimally invasive technique is to use a flexible and/or articulable elongate device, such as a flexible catheter that can be inserted into anatomic passageways and navigated toward a region of interest within the patient anatomy. Medical tools, such as biopsy or therapy instruments, may be deployed through the catheter to perform a medical procedure at the region of interest.

### SUMMARY

The following presents a simplified summary of various examples described herein and is not intended to identify key or critical elements or to delineate the scope of the claims.

An example of a medical tool may include a sheath and an elongate instrument. The sheath may include a tubular shaft member including a sheath channel, and a guard member attached to a distal portion of the tubular shaft member. The guard member may include a head portion, an elongate body sized for receipt within the sheath channel, and a lumen extending through the head portion and the elongate body. The elongate instrument may be sized for passage within the sheath channel and may include a flexible elongated tubular section having a body wall. The elongate instrument may further include a rigid tip extending from a distal end of the tubular section, the rigid tip including an opening, and a flexible jacket covering at least a portion of the body wall. The flexible jacket may include a chamfered distal end terminating proximally of the opening.

Another example of a medical tool may include a sheath and an elongate instrument. The sheath may include a tubular shaft member including a sheath channel, and a guard member attached to a distal portion of the tubular shaft member. The guard member may include a rigid distal section and a flexible proximal section. The elongate instrument may be sized for passage within the sheath channel may include a flexible elongated tubular section having a body wall including a plurality of slits. The elongate instrument may further include a rigid tip extending from a distal portion of the tubular section and a flexible jacket covering at least a portion of the tubular section. The flexible jacket may have a distal portion disposed distally of a proximal end of the guard member in both an extended configuration and a retracted configuration.

Another example of a medical tool may include a sheath and an elongate instrument. The sheath may include a tubular shaft member including a sheath channel, and a guard member attached to a distal portion of the tubular shaft member. A portion of the sheath channel adjacent a proximal end of the guard member may be tapered from a first diameter at a first location to a smaller second diameter at a second location disposed distally of the first location. The elongate instrument may be sized for passage within the sheath channel and may include a flexible elongated tubular section having a body wall including a plurality of slits. The elongate instrument may further include a rigid tip extending from a distal end of the tubular section and a flexible jacket covering at least a portion of the tubular section.

Another example of a medical tool may include a sheath having a tubular shaft member including a sheath channel, and a guard member attached to a distal portion of the tubular shaft member. The guard member may have a head portion including a plurality of flexible prongs defining a distal end of the guard member. A distal outer diameter of the distal end of the guard member may be smaller than an outer diameter of the tubular shaft member.

An example of a method of forming a chamfer in a flexible jacket may include: applying a flexible jacket to an elongate instrument sized for passage within a sheath channel; disposing a sleeve over an end of the flexible jacket; shrinking the sleeve by heating the sleeve; and removing the sleeve from the flexible jacket.

Another example method of forming a chamfer in a flexible jacket may include: applying a flexible jacket to an elongate instrument sized for passage within a sheath channel; placing a portion of the elongate instrument on which an end of the flexible jacket is disposed into a die; and compressing the end of the flexible jacket with the die while applying heat.

Yet another example method of forming a chamfer in a flexible jacket may include: applying a flexible jacket to an elongate instrument sized for passage within a sheath channel; and ablating a portion of an end surface of the flexible jacket.

An example method of forming an internally-tapered section in a sheath of a medical tool may include: securing a guard member to a distal portion of the sheath such that an elongate body of the guard member is disposed within a sheath channel of the sheath; inserting a mandrel into the sheath channel; and heating an internal wall of the sheath channel with the mandrel.

Another example of a method of forming an internally-tapered section in a sheath of a medical tool may include: securing a guard member to a distal portion of the sheath such that an elongate body of the guard member is disposed within a sheath channel of the sheath, and applying a laser to the sheath until a diameter of the sheath channel adjacent the distal end of the elongate body is reduced.

It is to be understood that both the foregoing general description and the following detailed description are illustrative and explanatory in nature and are intended to provide an understanding of the present description without limiting the scope of the present description. In that regard, additional aspects, features, and advantages of the present description will be apparent to one skilled in the art from the following detailed description.

### BRIEF DESCRIPTIONS OF THE DRAWINGS

FIG. 1A illustrates a medical tool including an elongate instrument having a flexible jacket.

FIG. 1B illustrates the elongate instrument of FIG. 1A.

FIG. 2A illustrates a medical tool including an elongate instrument having a flexible jacket with a tapered distal end and a guard member of a sheath.

FIG. 2B illustrates the elongate instrument of FIG. 2A.

FIG. 2C illustrates the guard member of FIG. 2A.

FIGS. 3A-3C are flowcharts of examples of a method for forming a chamfer in a flexible jacket.

FIG. 4 illustrates formation of a chamfer in of a distal end of a flexible jacket.

FIG. 5A illustrates a medical tool including a sheath having a guard member.

FIGS. 5B-5C illustrate the guard member of FIG. 5A.

FIG. 6 illustrates a medical tool including a sheath having a tapered inner diameter.

FIGS. 7A-7B are flowcharts of examples of methods for forming an internally-tapered section in a sheath

FIG. 8A illustrates a medical tool including a guard member having a plurality of flexible prongs, according to some examples.

FIGS. 8B-8C illustrate the guard member of FIG. 8A.

FIG. 8D illustrates a perspective view of a guard member having a plurality of flexible prongs, according to some examples.

FIG. 8E illustrates a cross sectional view of the guard member of FIG. 8D.

FIG. 8F illustrates a perspective view of a guard member having a plurality of flexible prongs, according to some examples.

FIG. 8G illustrates a cross sectional view of the guard member of FIG. 8F.

FIG. 9 is a simplified diagram of an example of a robotic-assisted medical system.

FIG. 10 is a simplified diagram of an example of a medical instrument system.

Examples of the present description and their advantages are best understood by referring to the detailed description that follows. It should be appreciated that like reference numerals are used to identify like elements illustrated in one or more of the figures, wherein showings therein are for purposes of illustrating examples of the present description and not for purposes of limiting the same.

### DETAILED DESCRIPTION

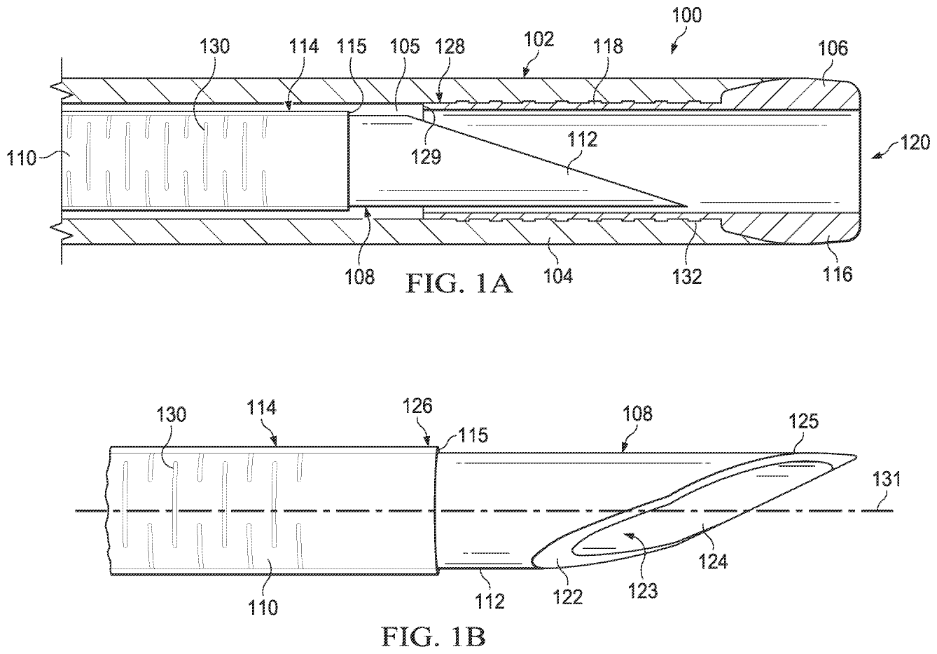

FIGS. 1A-1B illustrate a medical tool 100 including a sheath 102 and an elongate instrument 108. The sheath 102 may include a tubular shaft member 104 having a sheath channel 105 which extends along a length of the tubular shaft member 104. A guard member 106 may be fitted to a distal end of the tubular shaft member 104.

The guard member 106 may include a head portion 116 at its distal end and an elongate body 118 which extends proximally from the head portion 116. An outer circumference of the head portion 116 may be tapered toward a distal end and may have a rounded edge. The elongate body 118 may be sized for receipt within the tubular shaft member 104 and may include a plurality of ribs 132 around its circumference. A lumen 120 may extend through both the head portion 116 and the elongate body 118 of the guard member 106. The tubular shaft member 104 and/or the guard member 106 may include a radiopaque marker at any suitable location (not shown).

In the illustrated example, the elongate instrument 108 may be a biopsy needle. However, it should be appreciated that “elongate instrument” as used herein may refer to any instrument used for surgery, biopsy, ablation, illumination, irrigation, or suction, such as but not limited to, an endoscope, a laser, a therapeutic needle, etc.

The elongate instrument 108 may include a flexible tubular section 110 and a rigid tip 112. A body wall 122 may form a central lumen 123 which extends through an opening 124 at a distal end of the elongate instrument 108. The body wall 122 may be angled around the opening 124 to form a cutting edge 125. A plurality of slits 130 may be formed by laser-cutting or laser-etching of the body wall 122. The slits 130 may have a variety of circumferential configurations (as described below) that extend along the longitudinal length of the flexible tubular section 110. In the example illustrated in FIG. 1A, the slits 130 have a perpendicular slit pattern in which adjacent slits have a similar circumferential length but are alternated with a rotation of 90°. Each slit 130 is approximately perpendicular to the longitudinal axis 131 of the elongate instrument 108.

A flexible jacket 114 may extend around and may be coupled with the flexible tubular section 110 in a manner which covers the slits 130 or a portion thereof.

The elongate instrument 108 of the illustrated example may have approximately a 1 mm outer diameter. The sheath 102 may have approximately a 1.75 mm outer diameter. In one example, the length of rigid tip 112 may be approximately 5 mm, but longer or shorter rigid portions may be suitable. In other examples, smaller or larger sized elongate instruments may be used with the principles of this description.

The tubular shaft member 104 of the sheath 102 may be formed from a flexible material such as Pebax which may be mixed with a lubricious material. The guard member 106 may be made of a more rigid material than the tubular shaft member 104. Suitable material for the guard member 106 may include metal or hardened plastic. For example, the guard member 106 may comprise nitinol, stainless steel, or precipitation hardening stainless steel. The guard member 106 may comprise a radiopaque material that may be visualized on fluoroscopic images.

The elongate instrument 108 may be comprised of any suitable material. For example, the rigid tip 112 may be formed from a relatively rigid material including a metal, such as stainless steel, or a rigid polymer. The flexible tubular section 110 may generally be comprised of the same material as the rigid tip 112 or may be comprised of a different, more flexible material.

The flexible jacket 114 may be formed from, for example, a polymer material that adheres to and/or interlocks with the flexible tubular section 110 by extending into the slits 130. In one example, the flexible jacket 114 may be formed from a thin polyethylene terephthalate (PET) heat shrink material that may be molded onto the flexible tubular section 110. The heat shrink material may interlock with the flexible tubular section 110 by flowing into the slits 130 and when cooled, frictionally anchoring the flexible jacket 114 to the elongate instrument 108. In other examples, polyamide, polyimide, Pebax, polytetrafluorotheylene (PTFE), fluorinated ethylene propylene (FEP) and polyurethane may be used for the flexible jacket 114. Alternatively, a thermoplastic tubing such as Pebax with a low durometer (e.g. 35 D) may be molded (e.g., via thermal flow) into the slits 130, closing off the slits 130 to allow a vacuum, but flexible enough to allow bending. During the thermal flow, a mandrel with an outer diameter similar to the inner diameter of the central lumen 123 may be extended into the central lumen 123 to prevent the material from flowing into and obstructing the central lumen 123.

The sheath 102 may protect the distal end of the elongate instrument 108 from damage while being inserted through a catheter and may protect the internal surface of the catheter, or tissue where no catheter is used, from becoming damaged by a sharp tip and/or cutting edges of the elongate instrument 108. The sheath 102 may be positioned around the end of the elongate instrument 108 while the pair are advanced together through a catheter or anatomical passageway to a target anatomical location. Once the sheath 102 and elongate instrument 108 are fully advanced and positioned, the rigid tip 112 may be extended distally from the lumen 120 of the sheath 102. After use, the elongate instrument 108 may be retracted into the sheath 102, and the elongate instrument 108 and sheath 102 may be withdrawn from the patient. In some examples, the elongate instrument 108 may be configured to obtain biopsy samples. In such an instance, the elongate instrument 108 may be extended approximately 0.5 cm, 1 cm, 3 cm, 4 cm, or any other length from the distal end of the sheath 102 to expose the cutting edge 125 and opening 124 to capture a tissue sample in the central lumen 123.

The guard member 106 may prevent a point of the elongate instrument 108 from gouging the inner surface of the tubular shaft member 104 of the sheath 102 or otherwise becoming caught in the sheath 102. The rounded edge of the head portion 116 may prevent tissue injury as it is navigated through an anatomical passageway or alternatively to prevent damage to a catheter through which the medical tool 100 may be inserted. The guard member 106 may also provide an otherwise flexible sheath 102 with a stiffer portion which aids in guiding the elongate instrument 108 in a straight trajectory when advancing past the distal end of the sheath 102.

The one or more slits 130 of the flexible tubular section 110 may allow it to bend. The flexible jacket 114 may be impervious to fluid and may act as a flexible barrier to fluid flow through the slits 130. For example, if a vacuum is applied along the central lumen 123 of the elongate instrument 108 to pull tissue and bodily fluids in through the opening 124, the flexible jacket 114 may prevent flow of the tissue and fluids out of or into the central lumen 123 through the slits 130. The flexible jacket 114 may also provide reduced friction between an outer surface of the elongate instrument 108 and an inner surface of the sheath 102 as the elongate instrument 108 transitions from a retracted configuration in which the tip of the elongate instrument 108 is disposed within the lumen 120 of the guard member 106 (as illustrated in FIG. 1A) to an extended configuration in which the elongate instrument 108 protrudes from the sheath 102 and the tip of the elongate instrument 108 is disposed distally of the guard member 106.

In some examples, a proximal end 128 of the guard member 106 may interfere with a distal end 126 of the flexible jacket as the elongate instrument 108 transitions from the retracted configuration to the extended configuration. For example, the distal end 126 of the flexible jacket 114 may comprise a surface 115 which may be substantially transverse to the longitudinal axis 131. Similarly, the proximal end 128 of the guard member 106 may also comprise a surface 129 which may be generally transverse to the longitudinal axis 131. These two surfaces 115, 129 may collide and prevent the elongate instrument 108 from advancing distally in some instances. Such interference (e.g., snagging) may prevent or delay a medical procedure. In this regard, the examples described below may prevent or reduce interference between the flexible jacket 114 and the guard member 106.

Various alternatives are contemplated in regard to the above example. For example, with respect to slits 130, a single spiral slit may extend around the length of the flexible tubular section 110. Alternatively, an interrupted spiral slit pattern or an interrupted slit pattern, having a plurality of pitched or perpendicular slits (relative to the longitudinal axis 131), may be formed in the flexible tubular section 110. A continuous spiral pattern may allow deformable stretch or bend when the elongate instrument 108 navigates tight bends, but an interrupted spiral pattern or spaced arrangement may create bending flexibility while limiting linear deformation and stretch. The spacing of slits 130, width of slits 130, or angle of a spiral pattern, may vary to provide a desired flexibility. For example, the slit pattern may have approximately 2.5 cuts per rotation with 120° cut and 24° uncut with a slight pitch of approximately 0.006 inches.

Further, in alternative examples, in addition to or in place of the opening 124 at the distal end of the elongate instrument 108, an elongate instrument 108 may have a side opening through a lateral wall to collect sheared tissue biopsy samples.

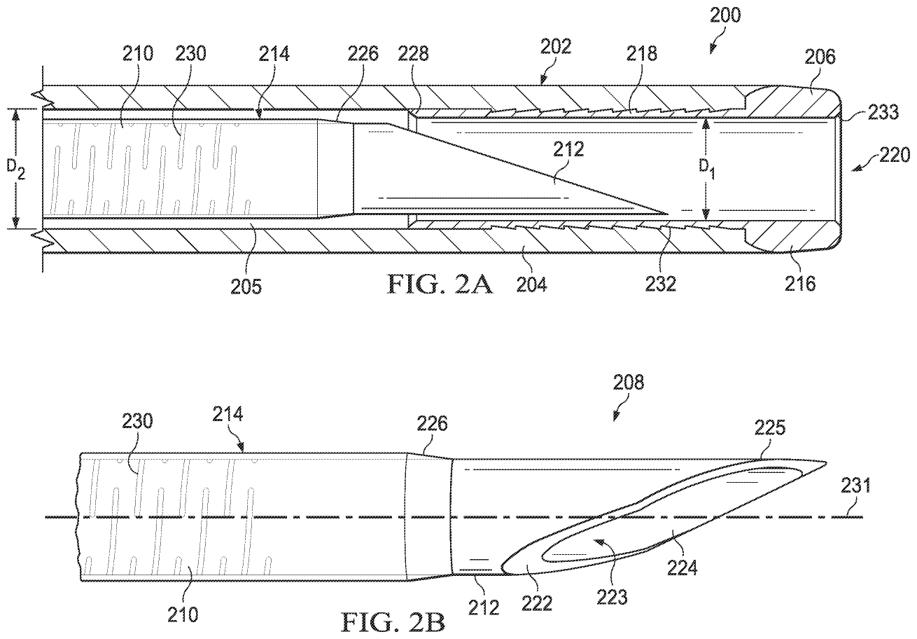

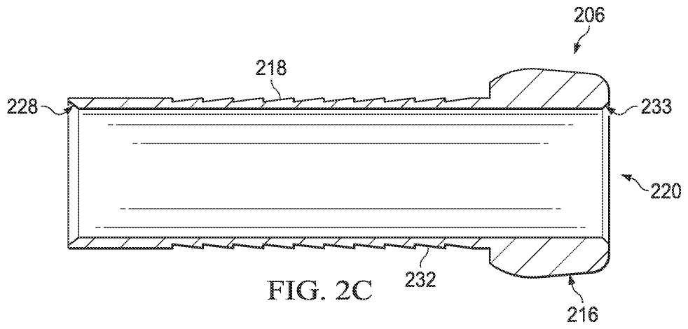

FIGS. 2A-2C illustrate an example of a medical tool 200 which may include a sheath 202 including a tubular shaft member 204 and a guard member 206 disposed at a distal end thereof. The tubular shaft member 204 may have a sheath channel 205 which extends along a length of the tubular shaft member 204.

The guard member 206 may include a head portion 216 at its distal end and an elongate body 218 that extends proximally from the head portion 216. The elongate body 218 may be sized for receipt within the tubular shaft member 204 and may include a plurality of barbs (e.g., annular sawteeth) 232 around its circumference for engagement with an inner surface of the tubular shaft member 204. An outer circumference of the head portion 216 may be tapered toward a distal end and may have rounded edges. The guard member 206 has a tapered opening 233 at the distal end of the lumen 220 with a profile that widens in a distal direction. Furthermore, a lumen 220 of the guard member 206 may include, at a proximal end 228 of the elongate body 218, a tapered profile widening in a proximal direction.

Similar to elongate instrument 108, the elongate instrument 208 may be a biopsy needle. The elongate instrument 208 may include a body wall 222 having an opening 224 into a central lumen 223. The body wall 222 is angled around the opening 224 to form a cutting edge 225. The elongate instrument 208 may include a flexible tubular section 210 and a rigid tip 212. The flexible tubular section 210 may include one or more slits 230 that extend through the wall of the elongate instrument 208 allowing it to bend. The slits 230 may have an interrupted spiral slit pattern, having a plurality of pitched slits 230 (relative to the longitudinal axis 231). A flexible jacket 214 extends around and is coupled with the flexible tubular section 210.

The tubular shaft member 204, guard member 206, elongate instrument 208, and flexible jacket 214 may each be formed from any suitable materials as discussed above with reference to FIG. 1.

As shown in FIG. 2A, elongate instrument 208 is disposed within the sheath 202. The elongate instrument 208 may be extended from the distal end of the sheath 202 to extract biopsy tissue. The tapered opening 233 may facilitate retraction of the elongate instrument 208 and prevent the guard member 206 from scraping or snagging the flexible jacket 214, as may occur with the squared lumen opening of guard member 106 of FIG. 1.

The profile of the lumen 220 at the proximal end 228 of the elongate body 218 may form a funnel-shaped opening into the guard member 206 which guides the elongate instrument 208 as it transitions from the retracted configuration to the extended configuration. In this regard, an inner diameter D₁ of the central lumen 220 may be smaller than an inner diameter D₂ of the sheath channel 205. The tapered proximal end 228 may aid the elongate instrument 208 in navigating the transition from D₂ to D₁.

The distal end 226 of the flexible jacket 214 may be tapered (e.g., chamfered or beveled) such that a thickness of the flexible jacket 214 reduces nearer the distal end 226. This chamfered shape may reduce the likelihood that the flexible jacket 214 snags on the guard member 206 as the elongate instrument 208 transitions from its retracted configuration within the sheath 202 to the extended configuration by reducing interference between the distal end 226 of the flexible jacket 214 and the proximal end 228 of the guard member 206. The tapered profile of the proximal end 228 of the guard member 206 further aids in preventing snagging of the flexible jacket 214 on the guard member 206. As shown in FIGS. 2A-B, both the proximal end 228 of the guard member 206 and the distal end 226 of the flexible jacket 214 have tapered profiles. In other examples, however, the proximal end 228 of the guard member 206 may have a tapered profile, but the distal end 226 of the flexible jacket 214 might not have a tapered profile. Alternatively, the distal end 226 of the flexible jacket 214 may have a tapered profile, but the proximal end 228 of the guard member 206 might not have a tapered profile in some examples.

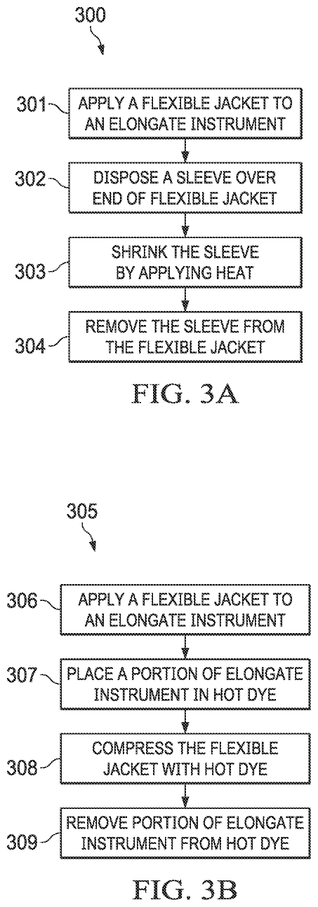

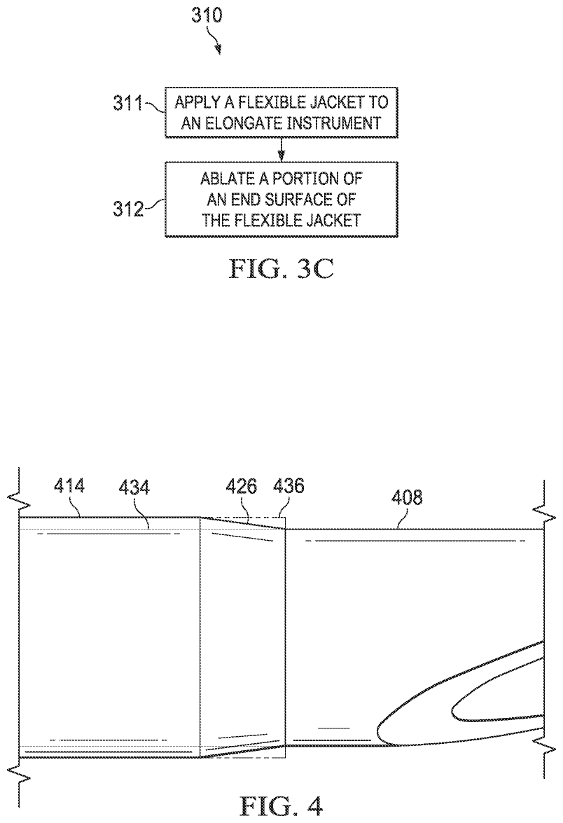

FIGS. 3A-3C illustrate methods of forming a chamfer in a flexible jacket of a medical tool, for example flexible jacket 214 of medical tool 200. Methods are illustrated herein as a set of operations or processes. Not all of the illustrated processes of any methods described herein may be performed in all examples of the respective method. Additionally, one or more processes that are not expressly illustrated in the figures may be included before, after, in between, or as part of the enumerated processes. In some examples, one or more of the processes are optional and may be omitted.

FIG. 3A illustrates an example of a method 300 for forming a chamfer in a flexible jacket of a medical tool. At a process 301, a flexible jacket may be applied to an elongate instrument. As an example, a sleeve of PET material may be slid over an elongate instrument (e.g., elongate instrument 208) with a plurality of slits. The PET material may be heat-shrunk until it is secured tightly around the elongate instrument. A portion of the PET material may protrude into the slits, further securing the flexible jacket of PET material to the elongate instrument. Application of a flexible jacket to the elongate instrument may yield a substantially squared distal end having a vertical surface. At a process 302, a sleeve may be disposed over the distal end of the flexible jacket. The sleeve may initially have a diameter which matches or exceeds a diameter of the flexible jacket such that the sleeve may be slid at least partially over the flexible jacket. A distal end of the sleeve may be aligned with the distal end of the flexible jacket or may extend distally across the distal end of the flexible jacket. The sleeve may be formed from any suitable material, for example, fluorinated ethylene propylene (FEP), polyether ether ketone (PEEK), or polytetrafluoroethylene (PTFE). At a process 303, the sleeve may be shrunk by applying heat to the sleeve in any suitable manner. Shrinking of the sleeve may compress the distal end of the flexible jacket, forming a chamfered edge (e.g., chamfered distal end 226 of flexible jacket 214). In one example, the portion of the elongate instrument supporting the sleeve may be placed in an oven or hot box at a suitable temperature to shrink the sleeve. In another example, heating the sleeve may include applying a laser to the sleeve for a sufficient period of time. The sleeve may be heated directly by the laser. Alternatively, the sleeve may be laser-clear such that the laser has a frequency that penetrates the sleeve and heats the flexible jacket such that the flexible jacket, in turn, heats the sleeve. In some or all of the example methods of applying heat to the sleeve, the flexible jacket may also be heated simultaneously with the sleeve to increase its malleability. The sleeve may remain positioned on the distal end of the flexible jacket until the flexible jacket has cooled and set. At a process 304, the sleeve may be removed from the flexible jacket by sliding or cutting it off of the elongate instrument. After the sleeve is removed, a chamfered edge (e.g., chamfered distal end 226 of flexible jacket 214) may remain.

FIG. 3B illustrates another example of a method 305 for forming a chamfer in a flexible jacket of a medical tool. At a process 306, a flexible jacket may be applied to an elongate instrument (e.g., elongate instrument 208), similar to process 301 of method 300. At a process 307, a portion of the elongate instrument supporting the flexible jacket may be placed into a die. The die may include two or more plates having recesses with a contoured interior shape which collectively define a desired chamfered shape of the distal end of the flexible jacket. At a process 308, the plates of the die may be closed around the portion of the elongate instrument to compress the flexible jacket. The die may be hot to soften the flexible jacket and increase its malleability. After a sufficient period of time has passed for reshaping the distal end of the flexible jacket into a chamfer (e.g., chamfered distal end 226 of flexible jacket 214), at a process 309, the portion of the elongate instrument may be removed from the die.

FIG. 3C illustrates yet another example of a method 310 for forming a chamfer in a flexible jacket of a medical tool. At a process 311, a flexible jacket may be applied to an elongate instrument (e.g., elongate instrument 208), similar to process 301 of method 300. At a process 312, a portion of the distal end of the flexible jacket may be ablated to form a chamfer (e.g., chamfered distal end 226 of flexible jacket 214), as described below with reference to FIG. 4. The process of ablation may be performed by laser, grinding, sanding, cutting, shaving, etc.

FIG. 4 illustrates forming a chamfer in a distal end of a flexible jacket 414. The flexible jacket 414 has a thickness extending between the outer surface 434 of the elongate instrument 408 and the outer surface of the flexible jacket 414. Dashed line 436 illustrates a distal end of flexible jacket 414 after application of the flexible jacket 414 to elongate instrument 408 and before forming a chamfer as described in methods 300, 305, and 310. As can be seen, a substantially vertical surface may define a distal end of the flexible jacket 414. Following the formation of a chamfer, the flexible jacket 414 may have a tapered profile as can be seen at the distal end 426.

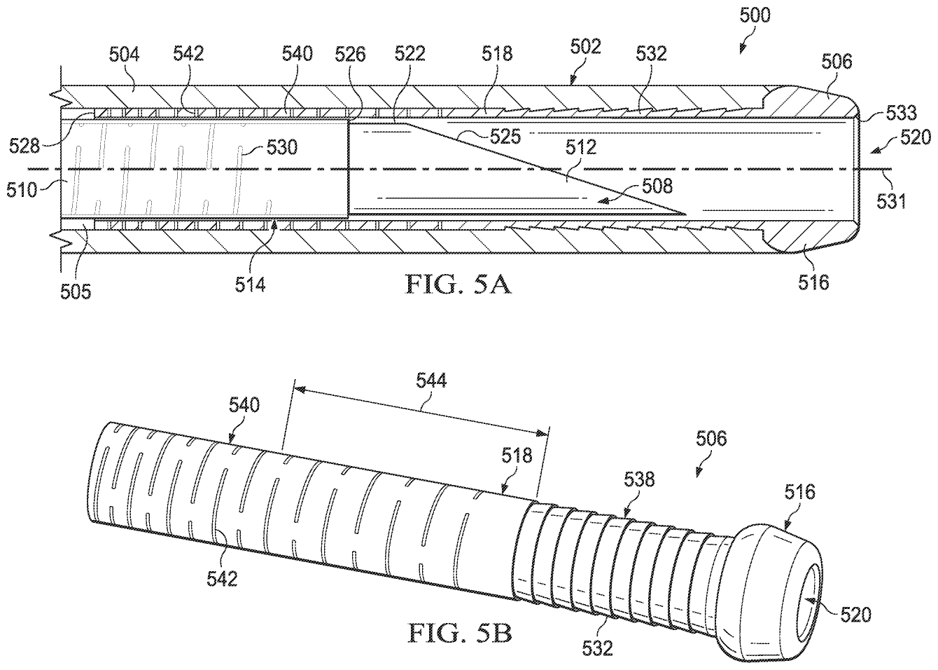

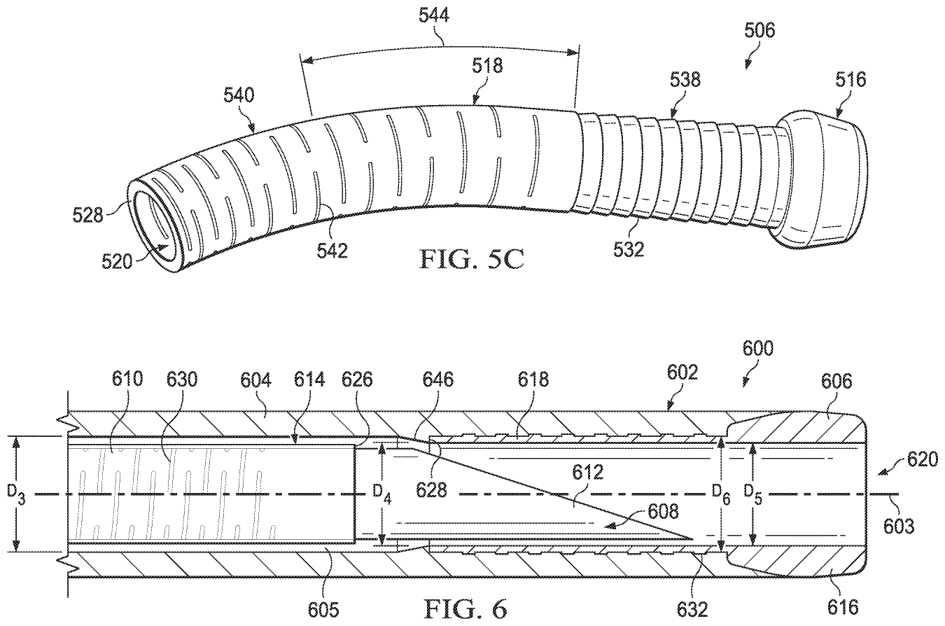

FIGS. 5A-5C illustrate an example of a medical tool 500 which may include a sheath 502 including a tubular shaft member 504 and a guard member 506 disposed at a distal end thereof. The tubular shaft member 504 may have a sheath channel 505 which extends along a length of the tubular shaft member 504.

The guard member 506 may include a head portion 516 at its distal end and an elongate body 518 extends proximally from the head portion 516. The elongate body 518 is sized for receipt within the tubular shaft member 504 and may include a plurality of barbs 532 around its circumference for engagement with an inner surface of the tubular shaft member 504. An outer circumference of the head portion 516 may be tapered toward a distal end and may have rounded edges. The guard member 506 has a tapered opening 533 at the distal end of the lumen 520 with a profile that widens in a distal direction. Furthermore, a lumen 520 of the guard member 506 may include, at a proximal end 528 of the elongate body 518, a tapered profile widening in a proximal direction.

Similar to elongate instrument 108, the elongate instrument 508 may be a biopsy needle. The elongate instrument 508 may include a body wall 522 having an opening into a central lumen (not shown). The body wall 522 is angled around the opening to form a cutting edge 525. The elongate instrument 508 may include a flexible tubular section 510 and a rigid tip 512. The flexible tubular section 510 may include one or more slits 530 that extend through the wall of the elongate instrument 508 allowing it to bend. The slits 530 may have an interrupted spiral slit pattern, having a plurality of pitched slits 530 (relative to the longitudinal axis 531). A flexible jacket 514 extends around and is coupled with the flexible tubular section 510.

The tubular shaft member 504, guard member 506, elongate instrument 508, and flexible jacket 514 may each be formed from any suitable materials as discussed above with reference to FIG. 1.

The elongate body 518 may include a rigid distal section 538 and a flexible proximal section 540 having a plurality of slits 542. A transition section 544 may extend between the flexible proximal portion 540 and the rigid distal section 538. This transition section 544 may be provided by spacing slits 542 according to a gradient with increased spacing between each successive distally adjacent slit 542. Alternatively, adjacent slits 542 in the transition section 544 may be at equal distances from each other, and the distance between adjacent slits 542 in the transition section 544 may be greater than the distance between adjacent slits 542 in the flexible proximal portion 540. The transition section 544 may be more rigid (or less flexible) than the flexible proximal portion, and/or the transition section 544 may be less rigid (or more flexible) than the rigid distal section 538. In addition to (or instead of) adjusting the distance between adjacent slits 542 in the transition section 544, the rigidity of the transition section 544 may be adjusted by increasing the width of the slits 542 to increase flexibility or decreasing the width of the slits 542 to increase rigidity. The rigid distal section 538 may include a plurality of barbs 532 around its circumference for engagement with an inner surface of the tubular shaft member 504. It is generally contemplated that the guard member 506 may have a length of approximately 6-50 mm, in which the rigid distal section 538 may have a length of approximately 3-30 mm. In one example, it is contemplated that the guard member 506 may have a length of approximately 8-10 mm, in which the rigid distal section 538 may have a length of approximately 5 mm. In this regard, “approximately” is intended to convey a range of at least+/−25%.

As shown in FIG. 5A, elongate instrument 508 is disposed within the sheath 502. The elongate instrument 508 may be extended from the distal end of the sheath 502 to extract biopsy tissue. The flexible proximal section 540 is configured to extend proximally beyond the distal end 526 of the flexible jacket 514 when the elongate instrument 508 is in the retracted configuration. In this regard, the distal end 526 of the flexible jacket 514 does not pass by the proximal end 528 of the guard member 506 when transitioning from the retracted configuration to the extended configuration, thereby avoiding snagging. While not shown in FIG. 5A, the distal end 526 of the flexible jacket 514 may also be chamfered, as previously discussed.

The slits 542 in the flexible proximal portion 540 allow that portion of the guard member 506 to flex along with the tubular shaft member 504 and the flexible tubular section 510 of the elongate instrument 508. Such flexibility provides for bending of the medical tool 500 as it traverses an anatomical passageway or a catheter. Providing a transition section 544 may prevent kinking and stress failure in the guard member 506 which can result from concentrating bending forces at an abrupt joint between a flexible portion and a rigid portion (e.g., no transition).

FIG. 6 illustrates an example of a medical tool 600 which may include a sheath 602 having a tubular shaft member 604 with a central sheath channel 605 and a guard member 606 disposed at a distal end thereof.

Similar to elongate instrument 108, the elongate instrument 608 may be a biopsy needle. The elongate instrument 608 may include a flexible tubular section 610 and a rigid tip 612. The flexible tubular section 610 may include one or more slits 630 that extend through the wall of the elongate instrument 608 allowing it to bend. A flexible jacket 614 may extend around and may be coupled with the flexible tubular section 610.

The guard member 606 may include a head portion 616 at its distal end and an elongate body 618 that extends proximally from the head portion 616. An outer circumference of the head portion 616 may be tapered toward a distal end and may have rounded edges. The elongate body 618 may be sized for receipt within the tubular shaft member 604 and include a plurality of ribs 632 around its circumference for engagement with an inner surface of the tubular shaft member 604.

The tubular shaft member 604, guard member 606, elongate instrument 608, and flexible jacket 614 may each be formed from any suitable materials as discussed above with reference to FIG. 1.

A tapered region 646 of the sheath channel 605 is tapered from a first diameter D₃ at a proximal portion of the sheath channel to a smaller second diameter D₄ adjacent a proximal end 628 of the guard member 606. An inner diameter D₅ of the guard member 606 in the lumen 620 may be substantially equal to the second diameter D₄. The second diameter D₄ may have any dimension between the outer diameter D₆ of the elongate body 618 and the inner diameter D₅ of the elongate body 618. In some examples, the second diameter D₄ may even be smaller than the inner diameter D₅ of the elongate body 618.

Elongate instrument 608 may be disposed within and extendable from the sheath 602. The elongate instrument 608 may be extended from the distal end of the sheath 602 to extract biopsy tissue. In this regard, a funnel-shaped surface formed in the tapered region 646 in the sheath channel 605 guides the elongate instrument 508 as it transitions from the retracted configuration to the extended configuration. By tapering radially inward toward the longitudinal axis 603 of the sheath 602, the tapered region 646 prevents the distal end 626 of the flexible jacket from snagging on the proximal end 628 of the guard member 606. While not shown in FIG. 6, the distal end 626 of the flexible jacket 614 may also be chamfered, as previously discussed.

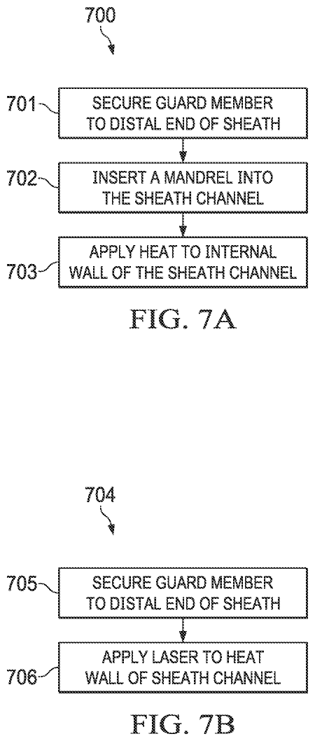

FIG. 7A illustrates a method 700 of forming an internally-tapered section in a sheath of a medical tool, for example, sheath 602 of medical tool 600. At a process 701, a guard member (e.g., guard member 606) may be secured to a distal end of a sheath (e.g., sheath 602), specifically, a tubular shaft member of a sheath. Ribs, barbs, or other protrusions on an external surface of an elongate body of the guard member may engage an inner surface of a sheath channel of the tubular shaft member. The tubular shaft member may be heated and shrunk onto the elongate body of the guard member and/or an adhesive may be applied to ensure bonding. At a process 702, a mandrel may be inserted into the sheath channel, either through the lumen of the guard member or through a proximal end of the tubular shaft member. A distal end of the mandrel may have a tapered outer diameter which generally corresponds to a desired shape of the tapered region to be formed, e.g., tapered region 646. At a process 703, heat may be applied to the internal wall of the sheath channel to soften or melt the tubular shaft member and cause it to take the tapered shape of the distal end of the mandrel, thereby forming the tapered region 646. In some examples, the mandrel may be heated to the desired temperature prior to insertion into the sheath channel. Alternatively, the mandrel may be heated after it has been inserted and positioned. In some examples, the tubular shaft member may be placed in an oven or hot box while the mandrel is positioned within the sheath channel.

FIG. 7B illustrates a method 704 of forming an internally-tapered section in a sheath of medical tool, for example, sheath 602 of medical tool 600. At a process 705, a guard member (e.g., guard member 606) may be secured to a distal end of a sheath (e.g., sheath 602), specifically a tubular shaft member of a sheath, in a manner similar to that of process 701 of method 700. At a process 706, a laser may be applied to heat the inner wall of the tubular shaft member until a diameter of the sheath channel adjacent the distal end of the elongate body is reduced. An optional process may be used before the applying the laser which may include inserting a mandrel into the sheath channel. The mandrel may limit the reduction in the diameter of the sheath channel to a desired amount. In some examples, the sheath may be laser-clear such that the laser has a frequency configured to pass through the sheath and heat the guard member and/or mandrel such that the guard member and/or mandrel heats the internal wall of the sheath channel.

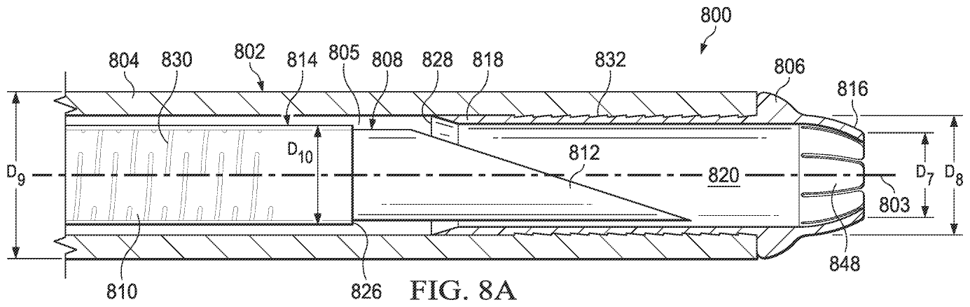

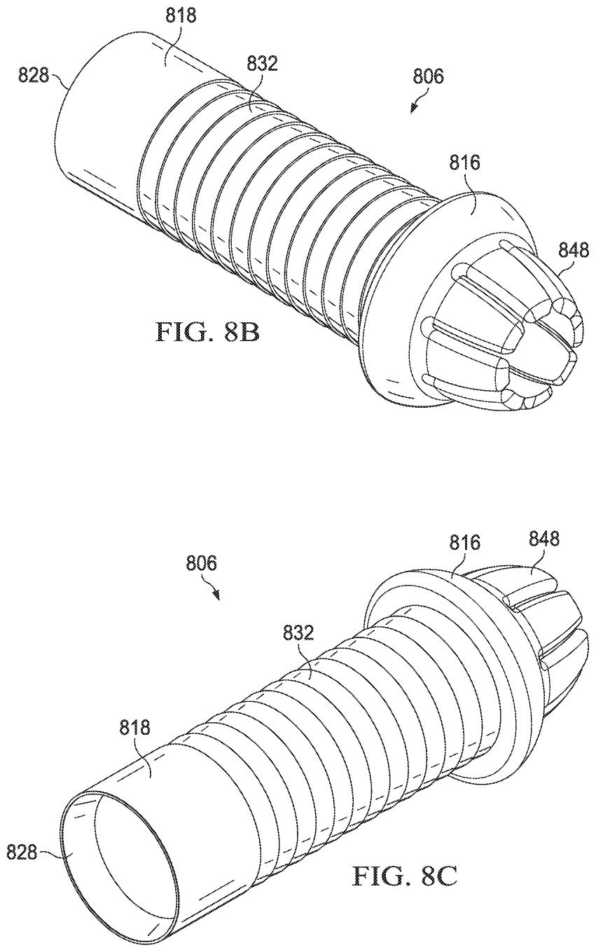

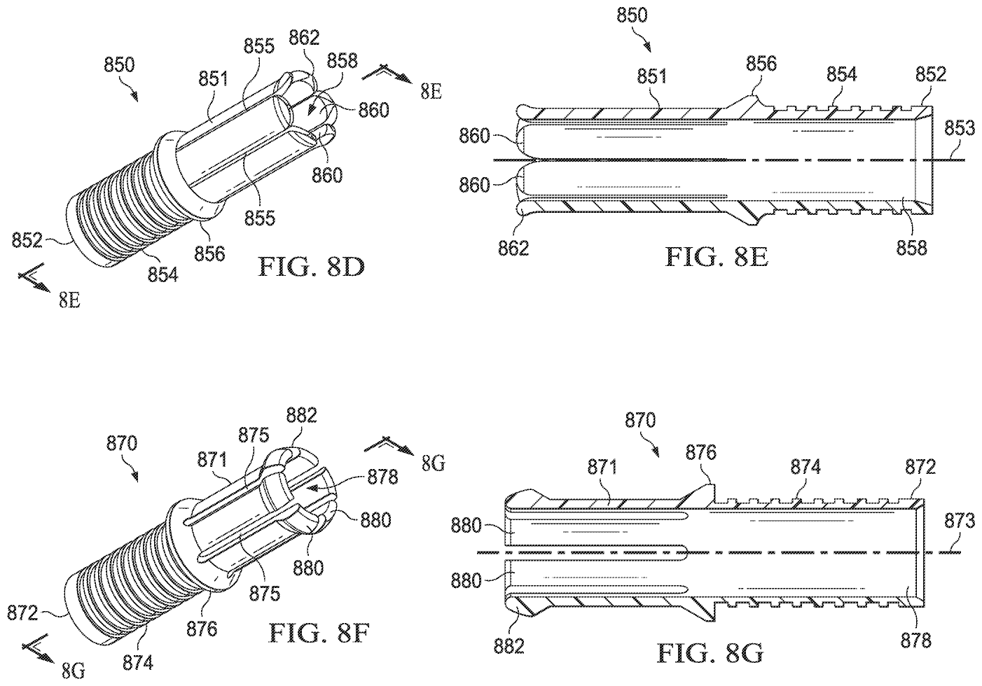

FIGS. 8A-8C illustrate an example of a medical tool 800 which may include a sheath 802 including a tubular shaft member 804 having a sheath channel 805 and a guard member 806 disposed at a distal end thereof.

Similar to elongate instrument 108, the elongate instrument 808 may be a biopsy needle. The elongate instrument 808 may include a flexible tubular section 810 and a rigid tip 812. The flexible tubular section 810 may include one or more slits 830 that extend through the wall of the elongate instrument 808. A flexible jacket 814 may extend around and may be coupled with the flexible tubular section 810.

The guard member 806 may include a head portion 816 at its distal end and an elongate body 818 which extends proximally from the head portion 816. The elongate body 818 may be sized for receipt within the tubular shaft member 804 and may include a plurality of barbs 832 around its circumference for engagement with an inner surface of the tubular shaft member 804. An outer circumference of the head portion 816 may be tapered toward a distal end and may have rounded edges. The lumen 820 of the guard member 806 may include, at a proximal end 828 of the elongate body 818, a tapered profile widening in a proximal direction.

The tubular shaft member 804, guard member 806, elongate instrument 808, and flexible jacket 814 may each be formed from any suitable materials as discussed above with reference to FIG. 1.

The head portion 816 of the guard member 806 may have a plurality of flexible prongs 848 defining a distal end of the guard member 806. An outer diameter D₇ of the guard member 806 at the distal end of the flexible prongs 848 may be approximately 5-50% smaller than an outer diameter D₈ of the guard member 806 at a proximal end of the flexible prongs 848. In one example, the guard member 806 at the distal end of the flexible prongs 848 may be approximately 10% smaller than an outer diameter of the guard member 806 at a proximal end of the flexible prongs 848. In this regard, an outer diameter D₇ of the distal end of the guard member 806 is smaller than an outer diameter D₉ of the tubular shaft member 804 such that the distal end of the sheath 802 may be narrower than a diameter D₉ of the sheath 802 along a majority of its length.

Each of the flexible prongs 848 may be inwardly flexible toward a longitudinal axis 803 of the sheath 802 in response to a force exerted on each of the flexible prongs 848 by a wall of a lumen through which the medical tool is insertable, such as in a catheter or anatomical passageway. The flexibility of the prongs 848 and the tapered or curved outer profile thereof may aid in traversing tight bends.

Furthermore, each of the flexible prongs 848 may be outwardly flexible away from a longitudinal axis 803 of the sheath 802 in response to a force exerted by an elongate instrument 808 extending from the sheath channel 805. For example, the outer diameter D₁₀ of the flexible jacket 814 may be larger than the outer diameter D₇ of the guard member 806 at the distal end of the flexible prongs 848 and/or may be larger than the inner diameter of the guard member 806 at the distal end of the flexible prongs 848. The shape and flexibility of the prongs 848 may prevent the guard member 806 from scraping or snagging the flexible jacket 814 as the elongate instrument 808 is retracted back into the sheath 802 after use. While not shown in FIG. 8A, the distal end 826 of the flexible jacket 814 may also be chamfered, as previously discussed.

FIG. 8D illustrates a perspective view of a guard member 850 including a head portion 851 at its distal end and an elongate body 852 which extends proximally from the head portion 851. FIG. 8E illustrates a cross-sectional view of the guard member 850. The elongate body 852 may be sized for receipt within a tubular shaft member (e.g., tubular shaft member 104) and may include a plurality of barbs 854 around its circumference for engagement with an inner surface of the tubular shaft member. The head portion 851 may include a proximal flange 856 that abuts a distal end of the tubular shaft member. A lumen 858 may extend through the guard member 850 and may include, at a proximal end of the elongate body 852, a tapered profile widening in a proximal direction. The tubular shaft member guard member 850 may be formed from any suitable materials, including stainless steel, as previously discussed for guard members.

The head portion 851 of the guard member 850 may include a plurality of flexible prongs 860 forming a distal end of the guard member 850. The flexible prongs may be separated by slits 855. In this example, the flexible prongs 860 may be arranged around and generally parallel to a longitudinal axis 853. Each of the flexible prongs 860 may be inwardly flexible with respect to the longitudinal axis 853 in response to a force exerted on the flexible prongs 860 by a wall of a lumen (such as in a catheter or anatomical passageway) through which the guard member 850 is inserted. Additionally, each of the flexible prongs 860 may be outwardly flexible with respect to the longitudinal axis 853 in response to a force exerted on the flexible prongs 860 by a medical tool (such as a needle) inserted through the lumen 858. The flexibility of the prongs 860 may, therefore, aid the otherwise relatively rigid guard member 850 in traversing tight bends through a delivery catheter and/or an anatomic passageway. Additionally, the prongs 860 may allow the guard member 850 to be sufficiently long to prevent exposure of or damage to a medical tool within the lumen 858, while also withstanding forces of friction that may compress and shorten the tubular shaft member (e.g., shaft member 104). In this example, the distal ends of the prongs 860 may include a flared end 862 extending away from the longitudinal axis 853. The flared end 862 may be rounded to prevent the guard member 850 from scraping or snagging the flexible jacket through which it is extended.

FIG. 8F illustrates a perspective view of a guard member 870 including a head portion 871 at its distal end and an elongate body 872 which extends proximally from the head portion 871. FIG. 8G illustrates a cross-sectional view of the guard member 870. The elongate body 872 may be sized for receipt within a tubular shaft member (e.g., tubular shaft member 104) and may include a plurality of barbs 874 around its circumference for engagement with an inner surface of the tubular shaft member. The head portion 871 may include a proximal flange 876 that abuts a distal end of the tubular shaft member. A lumen 878 may extend through the guard member 870 and may include, at a proximal end of the elongate body 872, a tapered profile widening in a proximal direction. The tubular shaft member guard member 870 may be formed from any suitable materials, including stainless steel, as previously discussed for guard members.

The head portion 871 of the guard member 870 may include a plurality of flexible prongs 880 forming a distal end of the guard member 870. The flexible prongs may be separated by slits 875. In this example, the flexible prongs 880 may be arranged around and generally parallel to a longitudinal axis 873. Each of the flexible prongs 880 may be inwardly flexible with respect to the longitudinal axis 873 in response to a force exerted on the flexible prongs 880 by a wall of a lumen (such as in a catheter or anatomical passageway) through which the guard member 870 is inserted. Additionally, each of the flexible prongs 880 may be outwardly flexible with respect to the longitudinal axis 873 in response to a force exerted on the flexible prongs 880 by a medical tool (such as a needle) inserted through the lumen 878. The flexibility of the prongs 880 may, therefore, aid the otherwise relatively rigid guard member 850 in traversing tight bends through a delivery catheter and/or an anatomic passageway. Additionally, the prongs 880 may allow the guard member 870 to be sufficiently long to prevent exposure of or damage to a medical tool within the lumen 878, while also withstanding forces of friction that may compress and shorten the tubular shaft member (e.g., shaft member 104). In this example, the distal ends of the prongs 860 may include a distal protuberance 882 to prevent the guard member 870 from scraping or snagging the flexible jacket through which it is extended. The slits 875 between the prongs 880 may be larger in this example than in the example of FIGS. 8D and 8E to allow a greater range of flexibility and prong movement.

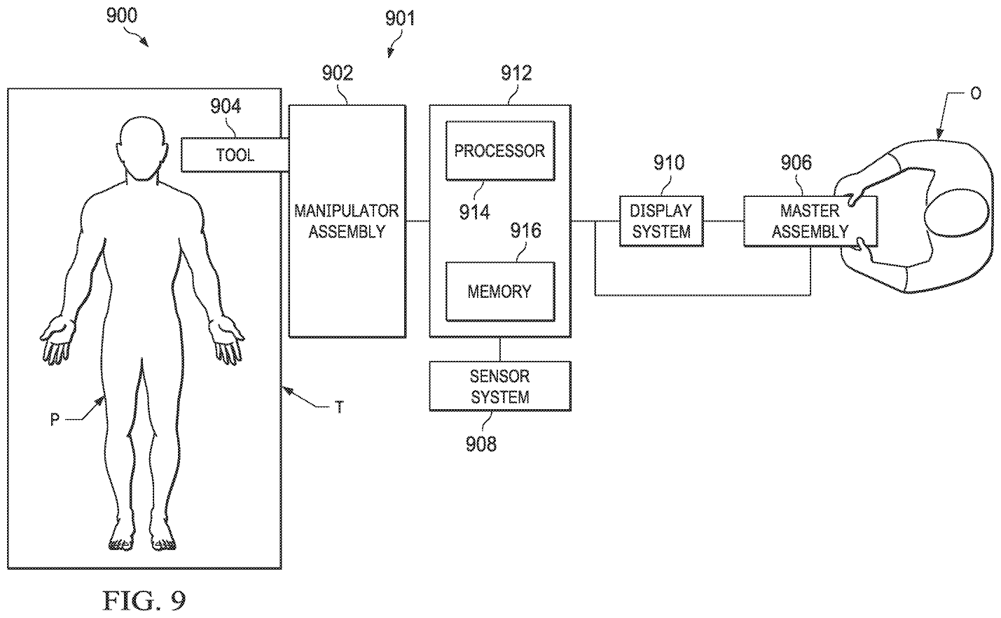

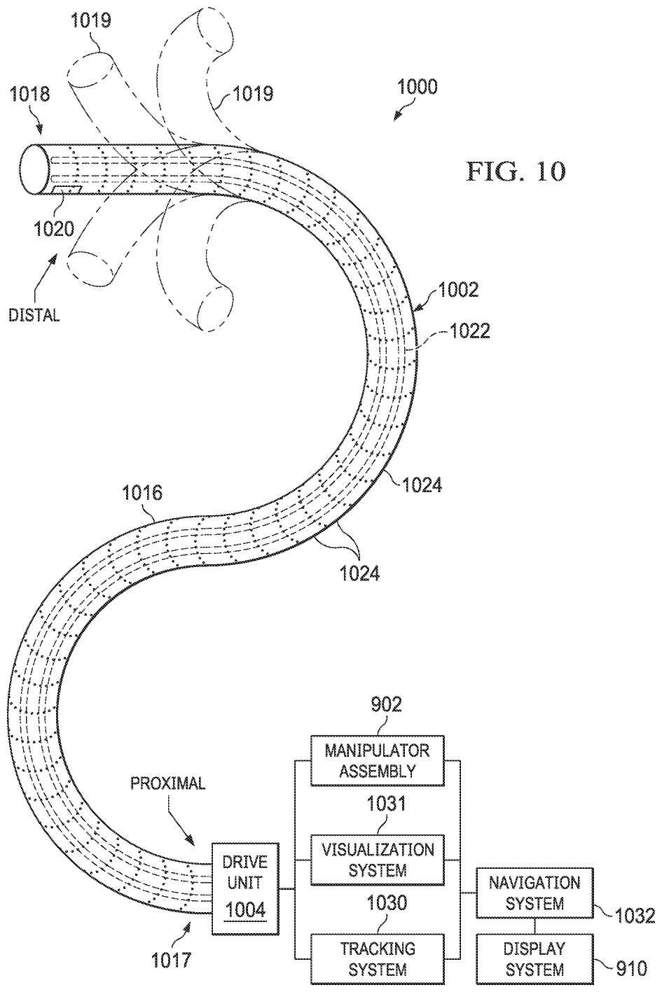

In some examples, the medical tools disclosed herein may be used in a medical procedure performed with a robotic-assisted medical system as described in further detail below. As shown in FIG. 9, a robotic-assisted medical system 900 may include a manipulator assembly 902 for operating a medical tool 904 in performing various procedures on a patient P positioned on a table T in a surgical environment 901. The medical tool 904 may correspond to any one of medical tool 100, 200, 500, 600, or 800, or any other medical tool within the scope of this description. Additionally or alternatively, the medical tool 904 may be a catheter having a lumen, as will be described in further detail with reference to FIG. 10. In these examples, any one of medical tool 100, 200, 500, 600, or 800 may be inserted into the lumen of the medical tool 904. The manipulator assembly 902 may be robotic-assisted, manually operated, or a hybrid assembly with select degrees of freedom of motion that may be motorized and/or select degrees of freedom of motion that may be non-motorized. A master assembly 906, which may be inside or outside of the surgical environment 901, generally may include one or more control devices for controlling manipulator assembly 902. Manipulator assembly 902 supports medical tool 904 and may include a plurality of actuators or motors that drive inputs on medical tool 904 in response to commands from a control system 912. The actuators may include drive systems that when coupled to medical tool 904 may advance medical tool 904 into a naturally or surgically created anatomic orifice. Other drive systems may move the distal end of medical tool in multiple degrees of freedom, which may include three degrees of linear motion (e.g., linear motion along the X, Y, Z Cartesian axes) and in three degrees of rotational motion (e.g., rotation about the X, Y, Z Cartesian axes). Additionally, the actuators can be used to actuate an articulable end effector of medical tool 904 for grasping tissue in the jaws of a biopsy device and/or the like.

Robotic-assisted medical system 900 also may include a display system 910 for displaying an image or representation of the surgical site and medical tool 904 generated by a sensor system 908 which may include an endoscopic imaging system. Display system 910 and master assembly 906 may be oriented so an operator O can control medical tool 904 and master assembly 906 with the perception of telepresence. Any of the previously described graphical user interfaces may be displayable on a display system 910 and/or a display system of an independent planning workstation.

In some examples, medical tool 904 may include components for use in surgery, biopsy, ablation, illumination, irrigation, or suction. Optionally medical tool 904, together with sensor system 908 may be used to gather (e.g., measure or survey) a set of data points corresponding to locations within anatomic passageways of a patient, such as patient P. In some examples, medical tool 904 may include components of the imaging system which may include an imaging scope assembly or imaging instrument that records a concurrent or real-time image of a surgical site and provides the image to the operator or operator O through the display system 910. In some examples, imaging system components may be integrally or removably coupled to medical tool 904. However, in some examples, a separate endoscope, attached to a separate manipulator assembly may be used with medical tool 904 to image the surgical site. The imaging system may be implemented as hardware, firmware, software or a combination thereof which interact with or are otherwise executed by one or more computer processors, which may include the processors of the control system 912.

The sensor system 908 may include a position/location sensor system (e.g., an electromagnetic (EM) sensor system) and/or a shape sensor system for determining the position, orientation, speed, velocity, pose, and/or shape of the medical tool 904.

Robotic-assisted medical system 900 may also include control system 912. Control system 912 may include at least one memory 916 and at least one computer processor 914 for effecting control between medical tool 904, master assembly 906, sensor system 908, and display system 910. Control system 912 also may include programmed instructions (e.g., a non-transitory machine-readable medium storing the instructions) to implement a plurality of operating modes of the robotic-assisted medical system including a navigation planning mode, a navigation mode, and/or a procedure mode. Control system 912 also may include programmed instructions (e.g., a non-transitory machine-readable medium storing the instructions) to implement some or all of the methods described in accordance with aspects disclosed herein, including, for example, moving a mounting bracket coupled to the manipulator assembly to the connection member, processing sensor information about the mounting bracket and/or connection member, and providing adjustment signals or instructions for adjusting the mounting bracket.

Control system 912 may further include a virtual visualization system to provide navigation assistance to operator O when controlling medical tool 904 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like.

FIG. 10 is a simplified diagram of a medical instrument system 1000 according to some examples. Medical instrument system 1000 may include elongate device 1002, which may be the same as or similar to medical tool 904 of FIG. 9 or any sheath described herein, coupled to a drive unit 1004. Elongate device 1002 may include a flexible body 1016 having proximal end 1017 and distal end or tip portion 1018. Medical instrument system 1000 further may include a tracking system 1030 for determining the position, orientation, speed, velocity, pose, and/or shape of distal end 1018 and/or of one or more segments 1024 along flexible body 1016 using one or more sensors and/or imaging devices as described in further detail below.

Tracking system 1030 may optionally track distal end 1018 and/or one or more of the segments 1024 using a shape sensor 1022. Shape sensor 1022 may optionally include an optical fiber aligned with flexible body 1016 (e.g., provided within an interior channel (not shown) or mounted externally). The optical fiber of shape sensor 1022 forms a fiber optic bend sensor for determining the shape of flexible body 1016. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389 (filed Jul. 13, 2005) (disclosing “Fiber optic position and shape sensing device and method relating thereto”); U.S. patent application Ser. No. 12/047,056 (filed on Jul. 16, 2004) (disclosing “Fiber-optic shape and relative position sensing”); and U.S. Pat. No. 6,389,187 (filed on Jun. 17, 1998) (disclosing “Optical Fibre Bend Sensor”), which are all incorporated by reference herein in their entireties. Sensors in some examples may employ other suitable strain sensing techniques, such as Rayleigh scattering, Raman scattering, Brillouin scattering, and Fluorescence scattering. In some examples, the shape of the elongate device may be determined using other techniques. For example, a history of the distal end pose of flexible body 1016 can be used to reconstruct the shape of flexible body 1016 over the interval of time. In some examples, tracking system 1030 may optionally and/or additionally track distal end 1018 using a position sensor system 1020. Position sensor system 1020 may be a component of an EM sensor system with position sensor system 1020 including one or more conductive coils that may be subjected to an externally generated electromagnetic field. Each coil of the EM sensor system then produces an induced electrical signal having characteristics that depend on the position and orientation of the coil relative to the externally generated electromagnetic field. In some examples, position sensor system 1020 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point. Further description of a position sensor system is provided in U.S. Pat. No. 6,380,732 (filed Aug. 11, 1999) (disclosing “Six-Degree of Freedom Tracking System Having a Passive Transponder on the Object Being Tracked”), which is incorporated by reference herein in its entirety.

Flexible body 1016 may include a channel sized and shaped to receive a medical instrument. In various examples, any of the antenna instruments and sheaths described above may be inserted through the channel of the flexible body 1016. For example, any one of medical tool 100, 200, 500, 600, or 800 may be inserted into the channel of the flexible body 1016. Medical instrument may include, for example, image capture probes, biopsy instruments, laser ablation fibers, and/or other surgical, diagnostic, or therapeutic tools. Medical instrument may be used with an imaging instrument (e.g., an image capture probe) also within flexible body 1016.

Flexible body 1016 may also house cables, linkages, or other steering controls (not shown) that extend between drive unit 1004 and distal end 1018 to controllably bend distal end 1018 as shown, for example, by broken dashed line depictions 1019 of distal end 1018. In some examples, at least four cables are used to provide independent “up-down” steering to control a pitch of distal end 1018 and “left-right” steering to control a yaw of distal end 1018. Steerable elongate devices are described in detail in U.S. patent application Ser. No. 13/274,208 (filed Oct. 14, 2011) (disclosing “Catheter with Removable Vision Probe”), which is incorporated by reference herein in its entirety.

The information from tracking system 1030 may be sent to a navigation system 1032 where it is combined with information from image processing system 1031 and/or the preoperatively obtained models to provide the operator with real-time position information. In some examples, the real-time position information may be displayed on display system 910 of FIG. 9 for use in the control of medical instrument system 1000. In some examples, control system 912 of FIG. 9 may utilize the position information as feedback for positioning medical instrument system 1000. Various systems for using fiber optic sensors to register and display a surgical instrument with surgical images are provided in U.S. patent application Ser. No. 13/107,562, filed May 13, 2011, disclosing, “Medical System Providing Dynamic Registration of a Model of an Anatomic Structure for Image-Guided Surgery,” which is incorporated by reference herein in its entirety.

In some examples, medical instrument system 1000 may be robotic-assisted within medical system 900 of FIG. 9. In some examples, manipulator assembly 902 of FIG. 9 may be replaced by direct operator control. In some examples, the direct operator control may include various handles and operator interfaces for hand-held operation of the instrument.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this description.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in this description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions. Not all the illustrated processes may be performed in all examples of the disclosed methods. Additionally, one or more processes that are not expressly illustrated in may be included before, after, in between, or as part of the illustrated processes. In some examples, one or more of the processes may be performed by a control system or may be implemented, at least in part, in the form of executable code stored on non-transitory, tangible, machine-readable media that when run by one or more processors may cause the one or more processors to perform one or more of the processes.

Any alterations and further modifications to the described devices, instruments, methods, and any further application of the principles of the present description are fully contemplated as would normally occur to one skilled in the art to which the description relates. In addition, dimensions provided herein are for specific examples and it is contemplated that different sizes, dimensions, and/or ratios may be utilized to implement the concepts of the present description. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for navigation and treatment of anatomic tissues, via natural or surgically created connected passageways, in any of a variety of anatomic systems, including the lung, colon, the intestines, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

One or more elements in examples of this description may be implemented in software to execute on a processor of a computer system such as control processing system. When implemented in software, the elements of the examples of this description may be code segments to perform various tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and/or magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples described herein are not described with reference to any particular programming language. It will be appreciated that a variety of programming languages may be used to implement the teachings of the examples described herein.

This application describes various instruments, portions of instruments, and anatomic structures in terms of their state in three-dimensional space. As used herein, the term position refers to the location of an object or a portion of an object in a three-dimensional space (e.g., three degrees of translational freedom along Cartesian x-, y-, and z-coordinates). As used herein, the term orientation refers to the rotational placement of an object or a portion of an object (e.g., in one or more degrees of rotational freedom such as roll, pitch, and/or yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term shape refers to a set of poses, positions, or orientations measured along an object.

While certain illustrative examples have been described and shown in the accompanying drawings, it is to be understood that such examples are merely illustrative of and not restrictive on the broad invention, and that the examples are not limited to the specific constructions and arrangements shown and described, since various other modifications may occur to those ordinarily skilled in the art.

What is claimed is:

1. A medical tool, comprising:
a sheath comprising:
a tubular shaft member including a sheath channel; and
a guard member attached to a distal portion of the tubular shaft member, the guard member comprising:
a head portion;
an elongate body sized for receipt within the sheath channel; and
a lumen extending through the head portion and the elongate body; and
an elongate instrument sized for passage within the sheath channel, the elongate instrument comprising:
a flexible elongated tubular section having a body wall;
a rigid tip extending from a distal end of the tubular section, the rigid tip including an opening; and
a flexible jacket covering at least a portion of the body wall, the flexible jacket including a chamfered distal end terminating proximally of the opening.

2. The medical tool of claim 1, wherein a proximal portion of the lumen has a tapered profile widening in a proximal direction.

3. The medical tool of claim 1, wherein a distal portion of the lumen has a tapered profile widening in a distal direction.

4. The medical tool of claim 1, wherein the body wall comprises a plurality of slits.

5. The medical tool of claim 4, wherein the flexible jacket extends into at least a portion of the plurality of slits.

6. The medical tool of claim 1, wherein an external surface of the elongate body comprises at least one circumferential rib or barb configured to engage a wall of the tubular shaft member defining the sheath channel.

7. A medical tool, comprising:
a sheath comprising:
a tubular shaft member including a sheath channel; and
a guard member attached to a distal portion of the tubular shaft member, wherein the guard member comprises a rigid distal section and a flexible proximal section; and
an elongate instrument sized for passage within the sheath channel, the elongate instrument comprising:
a flexible elongated tubular section having a body wall including a plurality of slits;
a rigid tip extending from a distal portion of the tubular section; and
a flexible jacket covering at least a portion of the tubular section, the flexible jacket having a distal portion disposed distally of a proximal end of the guard member in both an extended configuration and a retracted configuration.

8. The medical tool of claim 7, wherein the flexible proximal section of the guard member comprises a plurality of laser-etched slits.

9. The medical tool of claim 7, wherein the rigid distal section of the guard member comprises a ribbed or barbed outer surface.

10. The medical tool of claim 7, wherein the guard member has a length of approximately 8-10 mm, and wherein the rigid distal section of the guard member has a length of approximately 5 mm.

11. The medical tool of claim 7, wherein the guard member further comprises a transition section between the flexible proximal section and the rigid distal section, the transition section being more flexible than the rigid distal section and less flexible than the flexible proximal section.

12. The medical tool of claim 11, wherein the transition section of the guard member comprises a plurality of slits having varying spacing between adjacent slits forming a gradient along a length of the transition section.

13. The medical tool of claim 11, wherein the flexible proximal section of the guard member comprises a first plurality of slits and the transition section of the guard member comprises a second plurality of slits and wherein a spacing between adjacent slits of the second plurality of slits is greater than a spacing between adjacent slits of the first plurality of slits or a width of each slit of the second plurality of slits is less than a width of each slit of the first plurality of slits.

14. A medical tool, comprising:
a sheath comprising:
a tubular shaft member including a sheath channel; and
a guard member attached to a distal portion of the tubular shaft member, the guard member comprising a head portion including a plurality of flexible prongs defining a distal end of the guard member, wherein a distal outer diameter of the distal end of the guard member is smaller than an outer diameter of the tubular shaft member.

15. The medical tool of claim 14, wherein each of the flexible prongs is inwardly flexible toward a longitudinal axis of the sheath in response to a force exerted on each of the flexible prongs by a wall of a lumen through which the medical tool is insertable.

16. The medical tool of claim 14, wherein each of the flexible prongs is outwardly flexible away from a longitudinal axis of the sheath in response to a force exerted by an instrument extended from the sheath channel.

17. The medical tool of claim 14, wherein the guard member further comprises an elongate body extending proximally from the head portion, and wherein an external surface of the elongate body is ribbed or barbed to engage the tubular shaft member.

18. The medical tool of claim 14, further comprising an elongate instrument that comprises:
a flexible elongated tubular section;
a rigid tip extending from a distal end of the tubular section; and
a flexible jacket covering at least a portion of the flexible elongated tubular section, the flexible jacket including a chamfered distal end terminating at a location on the flexible elongated tubular section that is disposed proximal of a proximal end of the guard member when the elongate instrument is in a retracted configuration and is disposed distal of the proximal end of the guard member when the elongate instrument is in an extended configuration.

19. The medical tool of claim 14, wherein the guard member comprises a lumen, and wherein a proximal portion of the lumen has a tapered profile widening in a proximal direction.

20. The medical tool of claim 14, wherein a portion of the sheath channel adjacent a proximal end of the guard member is tapered from a first diameter at a location proximal of the guard member to a smaller second diameter at the proximal end of the guard member, wherein the second diameter corresponds to an inner diameter of a lumen of the guard member.
